(19) United States
(12) Patent Application Publication
(10) Pub. No.: US 2025/0127293 A1
(43) Pub. Date: Apr. 24, 2025

(54) SELF-FEED ORAL CLEANING APPLIANCE

(71) Applicant: Shenzhen Besman Electronic Technology Co.,LTD, Shenzhen (CN)

(72) Inventor: Xuewu Wang, Shenzhen (CN)

(21) Appl. No.: 19/001,459

(22) Filed: Dec. 25, 2024

(30) Foreign Application Priority Data
Nov. 6, 2024 (CN) 202411572551.5

(51) Int. Cl. A46B 15/00; A46B 5/00; A46B 13/02; A46B 17/06; A61C 17/22; A61L 2/10

(52) U.S. Cl. CPC A46B 15/0095; A46B 5/0095; A46B 13/02; A46B 17/065; A61C 17/222; A61L 2/10; A61C 2204/002; A61L 2202/11; A61L 2202/121; A61L 2202/122; A61L 2202/123; A61L 2202/16

(57) ABSTRACT

A self-feed oral cleaning appliance is provided, including a cleaning equipment storage box, cleaning equipment, a sterilization device, a power receiving mechanism, and a feed induction control circuit. A body of the cleaning equipment storage box of the self-feed oral cleaning appliance is not required to have a built-in storage battery. When the cleaning host unit is stored in the storage box, the feed induction control circuit and the power receiving mechanism cooperate with each other to apply power from the cleaning host unit to the sterilization device, thus achieving that the sterilization device sterilizes cleaning equipment in the cleaning equipment storage box. The manufacturing costs of the cleaning appliance with the sterilization device can be effectively reduced, and logistics transportation is also facilitated. The self-feed oral cleaning appliance is high in practicability and is also beneficial to assist in promotion of oral health undertakings.

### TECHNICAL FIELD

The present disclosure relates to the technical field of oral cleaning, and in particular, to a self-feed oral cleaning appliance.

### BACKGROUND

Cleaning the mouth is an important way to maintain oral health. It is inevitable to use various oral cleaning appliances to assist in the cleaning. Common oral cleaning appliances include an electric toothbrush, an oral irrigator, an ultrasonic cleaner, and the like. When these appliances are used, the cleaning efficiency can be improved, and the cleaning effect can be enhanced.

However, after oral cleaning is completed, some residual bacteria may be more or less adhered to these appliances. If these bacteria are not promptly disinfected, they will continue to grow and may contaminate the oral cavity at the next time the appliances are used, causing incalculable oral health hazards.

In view of the above phenomena, some cleaning appliances on the market may be equipped with sterilization devices. To achieve lightweight, a sterilization device is arranged in a cleaning equipment storage box. After a cleaning appliance is used, the cleaning appliance can be stored in the storage box, and the sterilization device may be powered by a battery arranged in the storage box, or may be powered by using a charging cable to connect a power input port of the storage box to an external power supply, so that the sterilization device can work normally to achieve sterilization on the cleaning appliance stored in the storage box.

However, this kind of device may a built-in battery, which will greatly increase the manufacturing costs of the product and impose various limitations on the logistics transportation of the product. Or, the device will not be provided with a battery, and the sterilization operation can be achieved by using an external power supply, which will bring great inconvenience to users. However, in any case, it is difficult to gain competitive advantage in the increasingly stimulating market. As a result, the cleaning appliance with its own sterilization device is not favored by consumers, and thus, this kind of product is not popularized and used. This is strictly not conducive to the promotion of oral health undertakings.

### SUMMARY

The present disclosure aims to provide a self-feed oral cleaning appliance, a body of the cleaning equipment storage box of which is not required to have a built-in storage battery. When a cleaning host unit is stored in the storage box, a feed induction control circuit and a power receiving mechanism cooperate with each other to apply power from the cleaning host unit to a sterilization device, thus achieving that the sterilization device sterilizes cleaning equipment in the cleaning equipment storage box. This can effectively reduce the manufacturing costs of the cleaning appliance with the sterilization device, and is also convenient for logistics transportation. The self-feed oral cleaning appliance is high in practicability and is also beneficial to assist in the promotion of oral health undertakings. The problems mentioned in the above background section are solved.

To achieve the above objectives, the present disclosure provides the following technical solution: A self-feed oral cleaning appliance includes: a cleaning equipment storage box, configured to store cleaning equipment, wherein the inside of the cleaning equipment storage box is partitioned into a plurality of storage cavities; the storage cavities at least include a host unit storage cavity and a cleaning head storage cavity; the cleaning equipment, including a cleaning host unit and a cleaning head, wherein a storage battery is arranged in the cleaning host unit; the cleaning head is configured to clean an oral cavity; the cleaning host unit is configured to bear and/or drive the cleaning head to perform a cleaning operation; a sterilization device, arranged on a wall surface of the cleaning head storage cavity and configured to sterilize the cleaning head; a power receiving mechanism, including a power sending end and a power receiving end, wherein the power sending end is arranged on the cleaning host unit; the power receiving end is arranged on the cleaning equipment storage box; the power receiving mechanism is configured to achieve circuit connection between the cleaning host unit and the sterilization device; and a feed induction control circuit, configured to control power-on start and stop and power-on duration of the power receiving mechanism.

Preferably, the cleaning equipment storage box is provided with an observation window at a position corresponding to the cleaning head storage cavity; the observation window penetrates through a housing of the cleaning equipment storage box; and a transparent panel is encapsulated on an outer end surface of the observation window.

Preferably, the cleaning equipment storage box includes a storage box upper cover and a storage box base that are rotationally connected to each other; an accommodating cavity is formed between the storage box upper cover and the storage box base; and the plurality of storage cavities are formed in the accommodating cavity.

Preferably, one side edge of the storage box upper cover and one side edge, which is in the same direction as the side edge of the storage box upper cover, of the storage box base are connected to each other in a flipping manner through a soft rubber strip, and a first magnetic suction member and a second magnetic suction member are respectively arranged in on one side edge, opposite to the soft rubber strip, of the storage box upper cover and one side edge, opposite to the soft rubber strip, of the storage box base; the first magnetic suction member and the second magnetic suction member are connected in a magnetic suction manner; the storage box upper cover includes an upper cover outer shell and an upper cover inner shell; and the storage box base includes a base inner shell and a base outer shell.

Preferably, the cleaning equipment is an electric toothbrush; the cleaning host unit is an electric toothbrush handle; the cleaning head is a toothbrush head detachably connected to the electric toothbrush handle; a storage battery is arranged in the electric toothbrush handle; the power sending end of the power receiving mechanism is arranged at a tail end of the electric toothbrush handle and is encapsulated in an electric toothbrush tail end cover; the power receiving end of the power receiving mechanism is arranged on a wall surface of one side, facing the electric toothbrush tail end cover, of the host unit storage cavity; and when the electric toothbrush handle is stored in the host unit storage cavity, the power sending end and the power receiving end are electrically connected to each other.

Preferably, the power receiving mechanism includes a wireless transmitting coil, a wireless receiving coil, and a circuit board assembly; the power sending end is the wireless transmitting coil; the power receiving end is the wireless receiving coil and the circuit board assembly; the power sending end is arranged on the cleaning host unit; the circuit board assembly of the power receiving end is arranged in a housing of the cleaning equipment storage box; the wireless receiving coil is arranged on a wall surface of one side, close to the wireless transmitting coil, of the host unit storage cavity; and when the cleaning host unit is stored in the host unit storage cavity, the wireless transmitting coil is electromagnetically connected to the wireless receiving coil in a wireless manner.

Preferably, the power receiving mechanism includes a male pin for electrode connection and a female base for electrode connection; the power transmission end is the female base for electrode connection; the power receiving end is the male pin for electrode connection; the female base for electrode connection is arranged on the cleaning host unit; the male pin for electrode connection is arranged on a wall surface of one side, close to the female base for electrode connection, of the host unit storage cavity; and when the cleaning host unit is stored in the host unit storage cavity, the male pin for electrode connection and the female base for electrode connection are electrically connected to each other.

Preferably, the power receiving mechanism is electrically connected to a power input port; and the power input port is arranged in a preset through hole of a housing of the cleaning equipment storage box and extends out from the through hole.

Preferably, the sterilization device is an ultraviolet (UV) germicidal lamp; the UV germicidal lamp is encapsulated on the wall surface of the cleaning head storage cavity; and a light emission direction is towards an internal region of the cleaning head storage cavity.

Compared with the prior art, the present disclosure has the beneficial effects below:

A body of the cleaning equipment storage box of the self-feed oral cleaning appliance is not required to have a built-in storage battery. When the cleaning host unit is stored in the storage box, the feed induction control circuit and the power receiving mechanism cooperate with each other to apply power from the cleaning host unit to the sterilization device, thus achieving that the sterilization device sterilizes cleaning equipment in the cleaning equipment storage box. The manufacturing costs of the cleaning appliance with the sterilization device can be effectively reduced, and logistics transportation is also facilitated. The self-feed oral cleaning appliance is high in practicability and is also beneficial to assist in promotion of oral health undertakings.

### BRIEF DESCRIPTION OF THE DRAWINGS

FIG. 1 is a structural exploded view of the present disclosure;

FIG. 2 is a reference diagram I of a use state of the present disclosure;

FIG. 3 is a reference diagram II of a use state of the present disclosure; and

FIG. 4 is a reference diagram III of a use state of the present disclosure.

In the drawings: 1: cleaning equipment storage box; 11: storage box upper cover; 111: upper cover outer shell; 112: first magnetic suction member; 113: upper cover inner shell; 114: observation window; 115: transparent panel; 12: storage box base; 121: base inner shell; 122: second magnetic suction member; 123: base outer shell; 2: cleaning equipment; 21: cleaning host unit; 211: electric toothbrush tail end cover; 22: cleaning head; 3: sterilization device; 41: wireless transmitting coil; 42: wireless receiving coil; 43: circuit board assembly; and 44; power input port.

### DETAILED DESCRIPTION OF THE EMBODIMENTS

The technical solutions in the embodiments of the present disclosure will be clearly and completely described below with reference to the drawings in the embodiments of the present disclosure, and it is obvious that the described embodiments are only a part of the embodiments of the present disclosure but not all of them. Based on the embodiments of the present disclosure, all other embodiments obtained by those of ordinary skilled in the art without creative efforts fall within the protection scope of the present disclosure.

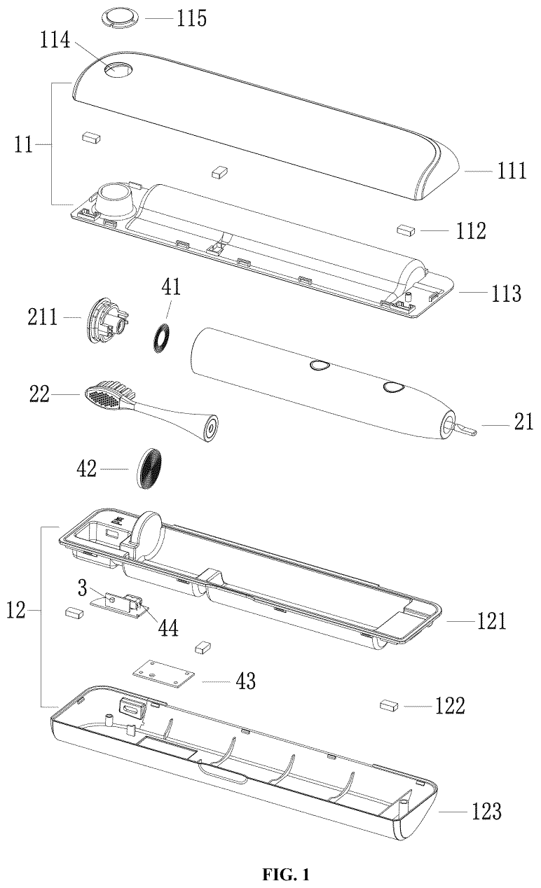

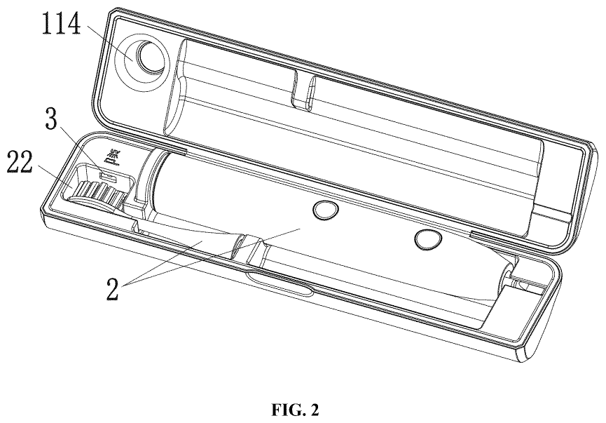

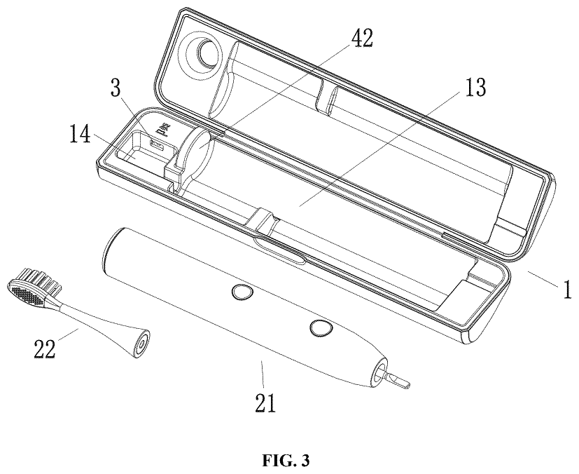

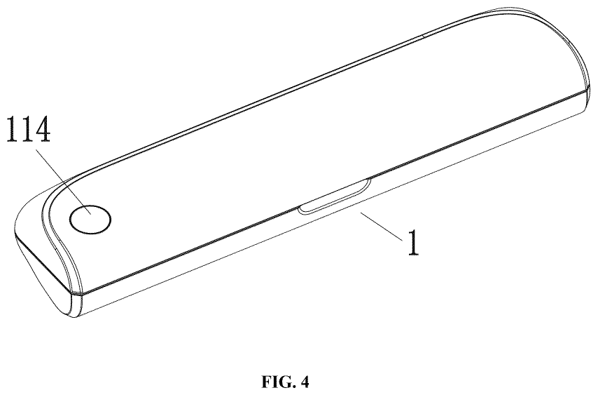

Referring to FIG. 1 to FIG. 4, a self-feed oral cleaning appliance includes: a cleaning equipment storage box 1, configured to store cleaning equipment 2, wherein the inside of the cleaning equipment storage box 1 is partitioned into a plurality of storage cavities; the storage cavities at least include a host unit storage cavity 13 and a cleaning head storage cavity 14; the cleaning equipment 2, including a cleaning host unit 21 and a cleaning head 22, wherein a storage battery is arranged in the cleaning host unit 21; the cleaning head 22 is configured to clean an oral cavity; the cleaning host unit is configured to bear and/or drive the cleaning head 22 to perform a cleaning operation; a sterilization device 3, arranged on a wall surface of the cleaning head storage cavity 14 and configured to sterilize the cleaning head 22; a power receiving mechanism, including a power sending end and a power receiving end, wherein the power sending end is arranged on the cleaning host unit 21; the power receiving end is arranged on the cleaning equipment storage box 1; the power receiving mechanism is configured to achieve circuit connection between the cleaning host unit 21 and the sterilization device 3; and a feed induction control circuit, configured to control power-on start and stop and power-on duration of the power receiving mechanism.

The cleaning equipment storage box 1 includes a storage box upper cover 11 and a storage box base 12 that are rotationally connected to each other; an accommodating cavity is formed between the storage box upper cover 11 and the storage box base 12; and the plurality of storage cavities are formed in the accommodating cavity. One side edge of the storage box upper cover 11 and one side edge, which is in the same direction as the side edge of the storage box upper cover, of the storage box base 12 are connected to each other in a flipping manner through a soft rubber strip, and a first magnetic suction member 112 and a second magnetic suction member 122 are respectively arranged in on one side edge, opposite to the soft rubber strip, of the storage box upper cover 11 and one side edge, opposite to the soft rubber strip, of the storage box base 12; the first magnetic suction member 112 and the second magnetic suction member 122 are connected in a magnetic suction manner; the storage box upper cover 11 includes an upper cover outer shell 111 and an upper cover inner shell 113; and the storage box base 12 includes a base inner shell 121 and a base outer shell 123. When the storage box upper cover 11 and the storage box base 12 are in a closed state, the first magnetic suction member 112 and the second magnetic suction member 122 can achieve an effect of keeping the closed state of the storage box upper cover 11 and the storage box base 12. The closed state between the storage box upper cover 11 and the storage box base 12 can be unlocked only when an external opening force is greater than a magnetic suction force.

In this embodiment, the cleaning equipment 2 is an electric toothbrush; the cleaning host unit 21 is an electric toothbrush handle; the cleaning head 22 is a toothbrush head detachably connected to the electric toothbrush handle; and a storage battery is arranged in the electric toothbrush handle. The power sending end of the power receiving mechanism uses a wireless transmitting coil 41 which is arranged at a tail end part of the electric toothbrush handle and is encapsulated in an electric toothbrush tail end cover 211. The power receiving end of the power receiving mechanism uses a wireless receiving coil 42 and a circuit board assembly 43. The wireless receiving coil 42 is arranged on a wall surface of one side, facing the electric toothbrush tail end cover 211, of the host unit storage cavity 13 and is close to the wireless transmitting coil 41. The circuit board assembly 43 is arranged in a housing of the cleaning equipment storage box 1. The wireless receiving coil 42 is electrically connected to the circuit board assembly 43. When the electric toothbrush handle is stored in the host unit storage cavity 13, the wireless transmitting coil 41 is electromagnetically connected to the wireless receiving coil 42 in a wireless manner.

The power receiving mechanism may further use a male pin for electrode connection and a female base for electrode connection for cooperation; the power transmission end is the female base for electrode connection; the power receiving end is the male pin for electrode connection; the female base for electrode connection is arranged on the cleaning host unit 21; the male pin for electrode connection is arranged on a wall surface of one side, close to the female base for electrode connection, of the host unit storage cavity 13; and when the cleaning host unit 21 is stored in the host unit storage cavity 13, the male pin for electrode connection and the female base for electrode connection are electrically connected to each other.

The power receiving mechanism is electrically connected to a power input port 44. The power input port 44 is arranged in a preset through hole of the housing of the cleaning equipment storage box 1 and extends out from the through hole. A charging cable is used to connect the power input port 44 to an external power supply to charge the storage battery in the cleaning host unit 21 to supplement power.

The sterilization device 3 is a UV germicidal lamp; the UV germicidal lamp is encapsulated on the wall surface of the cleaning head storage cavity 14; and a light emission direction is towards an internal region of the cleaning head storage cavity 14. The cleaning equipment storage box 1 is provided with an observation window 114 at a position corresponding to the cleaning head storage cavity 14; the observation window 114 penetrates through the housing of the cleaning equipment storage box 1; and a transparent panel 115 is encapsulated on an outer end surface of the observation window 114. Through the transparent panel 115, whether the UV germicidal lamp is lit can be observed, so as to determine whether the sterilization device 3 starts to work.

A working mode is as follows: when the electric toothbrush handle and the toothbrush head are respectively aligned with and stored in the host unit storage cavity 13 and the cleaning head storage cavity 14, the wireless receiving coil 42 is triggered by a magnetic field of the wireless transmitting coil 41. Under the action of a trigger signal, the feed induction control circuit in the circuit board assembly 43 is started, and a factory preset working mode is called to control the wireless receiving coil 42 and the wireless transmitting coil 41 to start a wireless power-on mode, and at the end of a set power-on duration disconnect, the circuit connection between the wireless receiving coil 42 and the wireless transmitting coil 41 is cut off. When the circuit connection is not cut off, current in the circuit can drive the UV germicidal lamp to be lit to sterilize the toothbrush head.

In summary, a body of the cleaning equipment storage box 1 of the self-feed oral cleaning appliance is not required to have a built-in storage battery. When the cleaning host unit 21 is stored in the storage box, the feed induction control circuit and the power receiving mechanism cooperate with each other to apply power from the cleaning host unit 21 to the sterilization device 3, thus achieving that the sterilization device 3 sterilizes cleaning equipment 2 in the cleaning equipment storage box 1. The manufacturing costs of the cleaning appliance with the sterilization device 3 can be effectively reduced, and logistics transportation is also facilitated. The self-feed oral cleaning appliance is high in practicability and is also beneficial to assist in promotion of oral health undertakings.

It should be noted that in this document, relationship terms such as first and second are used solely to distinguish one entity or operation from another entity or operation without necessarily requiring or implying any actual such relationship or order between such entities or operations. Furthermore, the terms “include”, “including”, or any other variation thereof, are intended to encompass a non-exclusive inclusion, such that a process, method, article, or device that includes a list of elements does not include only those elements but may include other elements not explicitly listed or inherent to such process, method, article, or device.

Although the embodiments of the present disclosure have been shown and described, it can be understood by those of ordinary skill in the art that various changes, modifications, substitutions, and variations can be made to these embodiments without departing from the principle and spirit of the present disclosure. The scope of the present disclosure is defined by the accompanying claims and their equivalents.

What is claimed is:

1. A self-feed oral cleaning appliance, comprising:
a cleaning equipment storage box (1), configured to store cleaning equipment (2), wherein the inside of the cleaning equipment storage box (1) is partitioned into a plurality of storage cavities; the storage cavities at least comprise a host unit storage cavity (13) and a cleaning head storage cavity (14);
the cleaning equipment (2), comprising a cleaning host unit (21) and a cleaning head (22), wherein a storage battery is arranged in the cleaning host unit (21); the cleaning head (22) is configured to clean an oral cavity; the cleaning host unit is configured to bear and/or drive the cleaning head (22) to perform a cleaning operation;
a sterilization device (3), arranged on a wall surface of the cleaning head storage cavity (14) and configured to sterilize the cleaning head (22);
a power receiving mechanism, comprising a power sending end and a power receiving end, wherein the power sending end is arranged on the cleaning host unit (21); the power receiving end is arranged on the cleaning equipment storage box (1); the power receiving mechanism is configured to achieve circuit connection between the cleaning host unit (21) and the sterilization device (3); and
a feed induction control circuit, configured to control power-on start and stop and power-on duration of the power receiving mechanism.

2. The self-feed oral cleaning appliance according to claim 1, wherein the cleaning equipment storage box (1) is provided with an observation window (114) at a position corresponding to the cleaning head storage cavity (14); the observation window (114) penetrates through a housing of the cleaning equipment storage box (1); and a transparent panel (115) is encapsulated on an outer end surface of the observation window (114).

3. The self-feed oral cleaning appliance according to claim 1, wherein the cleaning equipment storage box (1) comprises a storage box upper cover (11) and a storage box base (12) that are rotationally connected to each other; an accommodating cavity is formed between the storage box upper cover (11) and the storage box base (12); and the plurality of storage cavities are formed in the accommodating cavity.

4. The self-feed oral cleaning appliance according to claim 3, wherein one side edge of the storage box upper cover (11) and one side edge, which is in the same direction as the side edge of the storage box upper cover, of the storage box base (12) are connected to each other in a flipping manner through a soft rubber strip, and a first magnetic suction member (112) and a second magnetic suction member (122) are respectively arranged in on one side edge, opposite to the soft rubber strip, of the storage box upper cover (11) and one side edge, opposite to the soft rubber strip, of the storage box base (12); the first magnetic suction member (112) and the second magnetic suction member (122) are connected in a magnetic suction manner; the storage box upper cover (11) comprises an upper cover outer shell (111) and an upper cover inner shell (113); and the storage box base (12) comprises a base inner shell (121) and a base outer shell (123).

5. The self-feed oral cleaning appliance according to claim 1, wherein the cleaning equipment (2) is an electric toothbrush; the cleaning host unit (21) is an electric toothbrush handle; the cleaning head (22) is a toothbrush head detachably connected to the electric toothbrush handle; a storage battery is arranged in the electric toothbrush handle; the power sending end of the power receiving mechanism is arranged at a tail end of the electric toothbrush handle and is encapsulated in an electric toothbrush tail end cover (211); the power receiving end of the power receiving mechanism is arranged on a wall surface of one side, facing the electric toothbrush tail end cover (211), of the host unit storage cavity (13); and when the electric toothbrush handle is stored in the host unit storage cavity (13), the power sending end and the power receiving end are electrically connected to each other.

6. The self-feed oral cleaning appliance according to claim 1, wherein the power receiving mechanism comprises a wireless transmitting coil (41), a wireless receiving coil (42), and a circuit board assembly (43); the power sending end is the wireless transmitting coil (41); the power receiving end is the wireless receiving coil (42) and the circuit board assembly (43); the power sending end is arranged on the cleaning host unit (21); the circuit board assembly (43) of the power receiving end is arranged in a housing of the cleaning equipment storage box (1); the wireless receiving coil (42) is arranged on a wall surface of one side, close to the wireless transmitting coil (41), of the host unit storage cavity (13); and
when the cleaning host unit (21) is stored in the host unit storage cavity (13), the wireless transmitting coil (41) is electromagnetically connected to the wireless receiving coil (42) in a wireless manner.

7. The self-feed oral cleaning appliance according to claim 1, wherein the power receiving mechanism comprises a male pin for electrode connection and a female base for electrode connection; the power transmission end is the female base for electrode connection;
the power receiving end is the male pin for electrode connection; the female base for electrode connection is arranged on the cleaning host unit (21); the male pin for electrode connection is arranged on a wall surface of one side, close to the female base for electrode connection, of the host unit storage cavity (13); and when the cleaning host unit (21) is stored in the host unit storage cavity (13), the male pin for electrode connection and the female base for electrode connection are electrically connected to each other.

8. The self-feed oral cleaning appliance according to claim 1, wherein the power receiving mechanism is electrically connected to a power input port (44); and the power input port (44) is arranged in a preset through hole of a housing of the cleaning equipment storage box (1) and extends out from the through hole.

9. The self-feed oral cleaning appliance according to claim 1, wherein the sterilization device (3) is an ultraviolet (UV) germicidal lamp; the UV germicidal lamp is encapsulated on the wall surface of the cleaning head storage cavity (14); and a light emission direction is towards an internal region of the cleaning head storage cavity (14).
